(19) United States
(12) Patent Application Publication
(10) Pub. No.: US 2003/0204077 A1
(43) Pub. Date: Oct. 30, 2003

(54) RNA isolation reagent and methods

(75) Inventor: Domenica A. Simms, Silver Spring, MD (US)

Correspondence Address: STERNE, KESSLER, GOLDSTEIN & FOX PLLC, 1100 NEW YORK AVENUE, N.W., WASHINGTON, DC, 20005, US

(73) Assignee: Invitrogen Corporation

(21) Appl. No.: 10/442,946

(22) Filed: May 22, 2003

Related U.S. Application Data
(63) Continuation of application No. 09/058,350, filed on Apr. 10, 1998.
(60) Provisional application No. 60/041,837, filed on Apr. 10, 1997.

(51) Int. Cl. C07H 21/02; C23G 5/00

(52) U.S. Cl. 536/25.4; 252/364

(57) ABSTRACT

The invention provides methods, extraction reagents and kits for RNA isolation from eukaryotic cells such as plant or animal cells, where a phenol, a phenol solubilizer, a chelator and a nonionic detergent are used in the extraction reagent to replace the need for chaotropes and/or RNase inhibitors.

### BACKGROUND OF THE INVENTION

1. Field of the Invention

The present invention is in the field of molecular biology. The invention relates to improved methods, extraction reagents and kits for isolating RNA from eukaryotic cells, such as plant or animal cells.

2. Related Art

RNA isolation reagents and methods have been developed for isolating RNA, preferably cytoplasmic RNA and mRNA, from animal cells. These reagents and methods, when applied to the isolation of RNA from plant materials and plant cells, suffer from problems involving: the presence of Mg⁺⁺ ions which degrade RNA; co-isolation of polysaccharides with RNA when strong chaotropic salts or ionic detergents are used; and the use of high salt concentrations requiring additional precipitationlredissolving steps. These methods give unsatisfactory results when applied to the isolation of RNA from plants.

U.S. Pat. Nos. 5,346,994 and 4,843,155 disclose the use of extraction reagents having chaotropic salts (e.g., guanidinium isothiocyanate and ammonium isothiocyanate in high concentration (0.5-3M)), which completely disrupts cells and their nuclei, releasing RNA, DNA, proteins, membranous materials, and soluble polysaccharides into the extraction media. By virtue of the acidity and high salt concentration of the extraction media, the DNA precipitates out and is removed along with any insoluble cell debris during the centrifugation step.

Similar protocols are disclosed in Lewin, B. M., Genes III, John Wiley & Sons, publishers, New York, N.Y. (1990); Sambrook et al., Molecular Cloning: A Laboratory Manual, Second edition, Cold Spring Harbor Laboratory, Cold Spring Harbor, N.Y. (1989); and Ausubel et al., Current Protocols in Molecular Biology, Wiley Interscience, N.Y. (1987-1996).

The presence of chlorophyl and increased amounts of polysaccharides in plants provides additional problems for RNA isolation from such samples. In particular, the conventional use of chaotropic agents results in co-isolation of polysaccharides with the purified RNA. As plant cells have higher concentrations of polysaccharides, this problem is exacerbated by using known RNA isolation methods.

Furthermore, chlorophyl is broken down when plant cells are subjected to known RNA isolation methods and Mg⁺⁺ ions are released from the chlorophyl. The Mg⁺⁺ ions react with RNA and degrade it, reducing the yield and at least partially destroying the integrity of the RNA sequence.

Sambrook (supra, at sec. 7.6-7.9) discloses an alternative protocol for the isolation of cytoplasmic RNA from mammalian cells, which uses a non-disruptive RNA extraction reagent at physiological pH and salt concentration, instead of high concentrations of chaotropic salts. The extraction reagent contains an RNase inhibitor to protect the RNA during the isolation procedure and a nonionic detergent that solubilizes the cell membrane while leaving the nuclear membrane intact, to release cytoplasmic RNA. By not disrupting the nucleus and other cell organelles, the cytoplasmic RNA in the extraction media is selectively extracted from the cell, and separated from cell debris by a centrifugation step. However, the Sambrook protocol suffers from the problem of requiring expensive RNase inhibitors and extensive sample handling to recover the purified mRNA. The presence of a cell wall was also a deterrent from applying this technology to plant specimens.

Accordingly, improved methods and reagents are needed for RNA isolation from plants and plant or animal cells, which methods and reagents overcome one or more problems of known methods and reagents.

### SUMMARY OF THE INVENTION

The invention provides methods, extraction reagents and kits for RNA isolation from eukaryotic cells provided in a sample, where the use of at least a phenol, a chelator and a nonionic detergent replaces chaotropes and/or RNAse inhibitors in the extraction reagent. These methods, reagents and kits provide superior results for isolation of cytoplasmic RNA or mRNA from eukaryotic cells, especially from plants, maintaining the integrity of the RNA without co-isolation of polysaccharides.

In a method of the present invention, an extraction reagent is used on fresh or frozen eukaryotic cells, or preferably frozen, powdered plant and animal samples. The RNA is localized in the aqueous phase after phase separation, and then precipitated with alcohol.

The process can comprise:

(a) mixing the sample (or cell pellet) with the extraction reagent to form a mixture;

(b) adding a haloalkane to the mixture and mixing;

(c) separating the organic and aqueous phases by centrifugation or other known methods and transferring the aqueous phase; and

(d) precipitating the cytoplasmic RNA from the aqueous phase obtained in step (c) with alcohol.

The process can optionally further comprise: (e) recovering the cytoplasmic RNA from the precipitate obtained in step (d). The process can also optionally further comprise: (f) isolating mRNA from the recovered cytoplasmic RNA (e.g., as a precipitate) using any known method, such as by chromatography on oligo(dT)-cellulose (see, e.g., Sambrook, infra, at §§7.26-7.29).

The extraction reagent preferably comprises:

(a) at least one nonionic detergent (0.1-1.0% by volume);

(b) at least one chelator (0.02-0.25 M);

(c) at least one phenol (10%-60% by weight); and

(d) at least one phenol solubilizer (15%-55% by volume).

The extraction reagent optionally further comprises:

(e) at least one phenol stabilizer (0.05%-0.2% by weight);

The methods are useful for providing cytoplasmic or mRNA from cells contained in samples from eukaryotic organisms, with improved quantitative and/or qualitative yield over known methods, with RNA isolation from plants or plant cells preferred.

Another embodiment of the present invention relates to a kit comprising a carrier or receptacle being compartmentalized to receive and hold therein at least one container, wherein a first container contains at least one RNA reagent of the present invention, as described herein.

Other objects of the invention will be apparent to one of ordinary skill in the art from the following detailed description and examples relating to the present invention.

### DETAILED DESCRIPTION OF THE PREFERRED EMBODIMENTS

The invention is directed to methods and reagents using at least one of a phenol, a phenol stabilizer, a phenol solubilizer, a chelator and a nonionic detergent in the extraction reagent, in order to replace chaotropes and/or RNase inhibitors, and to provide enhanced isolation of intact cytoplasmic RNA from samples of eukaryotic organisms, tissues or cells.

In the present invention, subsequent sample handling is reduced substantially, as well as the material and labor costs for performing the RNA isolation. The use of a phenol, a chelator, a nonionic detergent, low salt concentrations, and no chaotropic agents improves the RNA isolation procedure, quantitatively and/or qualitatively.

The present invention provides several improvements over the related art, such as, but not limited to, at least one of: (a) the novel use of a chelator to protect the extracted RNA from degradation by Mg⁺⁺ ions (e.g., as present in animal cells, and as releasable from plant chlorophyl during the isolation process) in combination with phenol to protect the RNA from RNases; (b) the absence of strong chaotropic agents from the RNA extraction media minimizes co-isolation of polysaccharides with the purified RNA; (c) the use of the low salt concentration in the RNA extraction medium, where (after addition of chloroform and phase separation) mRNA can be selected directly from the aqueous phase, while reducing or eliminating additional precipitation/redissolving steps.

The methods and reagents of the invention provide isolation of cytoplasmic RNA from eukaryotic organisms, such as, but not limited to, one or more of plants or plant materials or cells, animal cells or tissue, insects or insect cells, fingal hyphae or cells and other nucleated cells.

In a preferred embodiment, the eukaryotic sample (e.g., a plant sample) is preferably ground to a powder in liquid nitrogen, or by any other known method, where the sample remains frozen throughout the grinding procedure before RNA isolation. Alternatively, cell samples can be fresh or frozen but need not be ground into a powder. The invention thus provides direct isolation of cytoplasmic RNA or mRNA from eukaryotic cells.

RNA Extraction Reagents

In a preferred embodiment, the extraction reagent comprises:

(a) at least one nonionic detergent (0.1-1.0% by volume) (e.g., tert-octylphenoxypoly(oxyethylene)ethanol (IGEPAL CA-630, Rhône Poulenc, France, 0.3-0.7%);

(b) at least one chelator (0.02-0.25 M) (e.g., 0.05-0.5 M sodium citrate); and

(c) at least one phenol (10%-60% by weight) (e.g., 2040%); and

(d) at least one phenol solubilizer (15%-55% by volume) (e.g., ethylene glycol, 22%).

The composition optionally further comprises:

(e) at least one phenol stabilizer (0.05%-0.2% by weight) (e.g., hydroxyquinoline, 0.1%).

Nonionic Detergents. Suitable nonionic detergents include, but are not limited to, at least one selected from the group consisting of adducts of ethylene oxide and fatty alcohols, alkyl phenols, and fatty acid amides, N,N-bis(3-D-gluconamidopropyl)cholamides (BIGCHAP), decanoyl-N-methylglucamides, n-decyl α-D-glucopyranosides, n-decyl β-D-glucopyranosides, n-decyl β-D-maltopyranosides, deoxy-BIGCHAPs, digitonins, n-dodecyl β-D-glucopyranosides, n-dodecyl α-D-maltosides, n-dodecyl β-D-maltosides, heptanoyl-N-methylglucamides, n-heptyl β-D-glucopyranosides, N-heptyl β-D-thioglucopyranosides, n-hexyl β-D-glucopyranosides, 1-monooleoyl-rac-glycerols, nonanoyl-N-methylglucamides, n-nonyl α-D-glucopyranosides, n-nonyl β-D-glucopyranosides, octanoyl-N-methylglucamides, n-octyl α-D-glucopyranosides, n-octyl β-D-glucopyranosides, octyl β-D-thiogalactopyranosides, octyl β-D-thioglucopyranosides, polyoxyethylene esters, polyoxyethylene ethers, octylphenoxypoly(oxyethylene)ethanol (IGEPAL CA-360, Triton X-100), polyoxyethylenesorbitan esters (Tween 20), sorbitan esters, tergitols, n-tetradecyl β-D-maltosides, tritons, tyloxapol, and n-undecyl β-D-glucopyranosides. Tritons can include, but are not limited to, triton X-100 (t-octylphenoxypolyethoxyethanol); triton X-100, peroxide- and carbonyl-free; triton X-100, reduced; triton X-100, reduced, peroxide- and carbonyl-free; triton X-114, triton X-405, triton N-101, triton X405, reduced; other tritons, such as but not limited to the following:

| Non-ionic | Low-foam |
|---|---|
| N-42 | CF-10 |
| N-57 | CF-21 |
| N-60 | CF-32 (95%) |
| X-15 | CF-54 |
| X-35 | DF-12 |
| X-45 | DF-16 |
| X-102 | |
| X-155 | |
| X-165 (70%) | |
| X-207 | |
| X-305 (70%) | |
| X-705-70 (70%) | |
| B-1956 (77%) | |

Additional suitable nonionic detergents include triton CG-110, triton XL-80N, triton WR-1339 or tyloxapol, tert-octylphenoxy poly(oxyethylene) ethanol (e.g., IGEPAL (Rhône-Poulenc, Paris, France)), and Nonidet P40 (octylphenoxy polyethoxy ethanol).

Chelators. Chelators include, but are not limited to: EDTAs, EGTAs, sodium citrates (citric acids), salicylic acids (and their salts), phthalic acids, 2,4-pentanediones, histidines, histidinol dihydrochlorides, 8-hydroxylquinolines, 8-hydroxyquinoline citrates, and o-hydroxyquinones.

Phenols. Any suitable phenolic compound can be used, e.g., according to the following formula I:

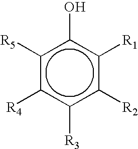

where R₁, R₂, R₃, R₄, R₅ are each independently selected from H, alkyl, halo, o-alkyl, acyl and hydroxyl. Examples include, but are not limited to: phenol, o-cresol, m-cresol, p-cresol, resorcinol, β-resorcylaldehyde and the like.

Phenol Stabilizers. Phenol stabilizers include, but are not limited to: 8-hydroxyquinolines, 8-hydroxyquinoline citrates, 2,5,7,8-tetramethyl-2-(4′,8′,12′-trimethyltridecyl)-6-chromanols, p-hydroxyquinones, o-hydroxyquinones, citric acids (and their salts), salicylic acids, ascorbic acids, p-phenylenediames, n-propylgallates, and other known radical scavengers.

Phenol Solubilizers. Phenol solubilizers include, but not limited to: any alcohol miscible with phenol and water, e.g., monoalcohols (such as, but not limited to: methyl alcohol, ethyl alcohol, propyl alcohol, and the like); diols (such as, but not limited to: ethylene glycol, propanediol, and the like); and, polyols (such as, but not limited to: glycerol, polyethylene glycol, polyvinyl alcohol, and the like).

The above compounds are commercially available, e.g., from Sigma-Aldrich (St. Louis, Mo.).

See, e.g., Myers, Surfactant Science and Technology, VCH Publishers, Inc., New York (1992); Cutler, supra; Rosen, Surfactants and Interfacial Phenomena, Wiley, New York (1984); Schick et al. Surfactant Science Series, Vols. 1-22, Dekker, New York (1961-1987); Tadros, Surfactants, Academic Press, London (1984); which references are entirely incorporated herein by reference with regard to their teaching of detergents and/or surfactants.

RNA Recovery Process

An example of a process according to the present invention for providing RNA, includes, but is not limited to:

(a) mixing the sample with the extraction reagent to form a mixture;

(b) adding a haloalkane to the mixture;

(c) separating the organic and aqueous phases; and

(d) precipitating the cytoplasmic RNA from the aqueous phase obtained in step (c).

The process can optionally further comprise: (e) recovering the cytoplasmic RNA from the precipitate obtained in step (d). The process can also optionally further comprise: (f) isolating mRNA from the recovered cytoplasmic RNA precipitate using any known method, such as by chromatography on oligo(dT)-cellulose (see, e.g., Sambrook, infra, at §7.26-7.29).

In step (b) of the above method, the haloalkane added to the sample and extraction reagent mixture can be any haloalkane suitable for separating RNA from other cytoplasmic components. Such haloalkanes include, but are not limited to chloroform, methylene chloride (dichloromethane), 1-bromo-3-chloro-propane, 2-bromo-1-chloropropane, bromoethane, 1-bromo-5-chloropentane, and bromotoluene.

In step (c) of the above method, the phases can be separated using any suitable method for separating RNA in an aqueous phase from other cytoplasmic components in the organic phase. Such separation methods include, but are not limited to centrifugation, filtration, vacuum filtration, or gravity.

In step (d) of the above method, the cytoplasmic RNA can be separated using any suitable method for precipitating RNA from the aqueous phase. Such separation methods include, but are not limited to using alcohol, polyethylene glycol, lithium chloride, or the like.

In step (e) of the above method, the cytoplasmic RNA can be recovered using any suitable method for recovering RNA from the precipitate. Such separation methods include, but are not limited to dissolving the RNA in water or in low salt buffer.

Having now generally described the invention, the same will be more readily understood through reference to the following example which is provided by way of illustration, and is not intended to be limiting of the present invention.

### EXAMPLE 1

### Isolation of Plant RNA

Protocol for Isolation of Cytoplasmic RNA from a Plant Sample or Specimen

The plant specimen is ground into a powder in liquid nitrogen. The plant powder is stored and used frozen. 0.1 g of the frozen powdered plant is thoroughly mixed with 1 ml of the RNA extraction reagent of the invention and is then let stand at room temperature for 5 minutes. 0.2 ml of chloroform per ml of the RNA extraction reagent is added to the mixture, and mixed thoroughly. The sample is centrifuged for 5 minutes at 12,000×g at room temperature. (Larger quantities can be centrifuged for 30 minutes at 2600×g or 6000×g for 10 minutes at 4° C.)

The aqueous phase is transferred to an RNase-free tube. 0.5 ml of isopropanol per ml of RNA extraction reagent is added. The sample is mixed and let stand at room temperature for 10 minutes. The sample is centrifuged for 10 minutes at 12,000×g at 4° C. (Larger quantities can be centrifuged for 30 minutes at 26000×g or 6000×g for 10 minutes at 4° C.) The supernate is decanted. The pellet is then washed with 1 ml of 75% ethanol per ml of RNA extraction reagent, centrifuged and any residual liquid is removed. RNase-free water is added to dissolve the RNA (50 μl of water for the 0.1 g sample).

Isolation of cytoplasmic RNA from cells grown in suspension. The cells are collected by centrifugation at 4° C. for 5 minutes at 2000 g. The fresh or frozen cell pellet containing 1×10⁷ cells is gently resuspended in 1 ml of RNA extraction reagent. The mixture is allowed to stand at room temperature for 5 minutes.

0.2 ml of chloroform is added per ml of RNA extraction reagent used., and mixed thoroughly. The sample is centrifuged for 5 minutes at 12,000×g at room temperature. (Larger quantities can be centrifuged for 30 minutes at 2600×g or 6000×g for 10 minutes at 4° C.) The top, aqueous phase is transferred to an RNase-free tube, and 0.5 ml of isopropanol is added per ml of RNA extraction reagent. The supernatant is mixed and let stand at room temperature for 10 minutes.

The sample is centrifuged for 10 minutes at 12,000×g at 4° C. (Larger quantities can be centrifuged for 30 minutes at 2600×g or 6000×g for 10 minutes at 4° C.) The supemate is decanted. It is then washed with 1 ml of 75% ethanol per ml of RNA extraction reagent, centrifuged and any residual liquid is removed. RNase-free water is added to dissolve the RNA (50 μl of water for the 1×10⁷ cells sample).

The concentration of the RNA is determined by measuring the OD₂₆₀ of an aliquot of the final preparation. A solution of RNA whose OD₂₆₀=1 contains approximately 40 μg of RNA per milliliter.

Results Using the Above Protocol.

The following Table 1 shows results obtained using the above protocol, as RNA yields.

TABLE I

| Sample | Method | Amount (g) | Ratio A_{260/280} | RNA (μg) |
|---|---|---|---|---|
| Tobacco leaves | RNA | 0.10 | 2.0 | 15 |
| (soil grown plant) | reagent | | | |
| Tobacco leaves | RNA | 1.0 | 1.7 | 212 |
| (soil grown plant) | reagent | | | |
| Tobacco leaves | RNA | 1.0 | 1.8 | 191 |
| (soil grown plant) | reagent | | | |
| Tobacco leaves | Trizol | 1.0 | 1.8 | 181 |
| (soil grown plant) | | | | |
| Tomato plant | RNA | 0.11 | 1.9 | 38 |
| (culture) | reagent | | | |
| Tomato plant | RNA | 1.0 | 1.9 | 351 |
| (culture) | reagent | | | |
| Tomato plant | Trizol | 0.8 | 1.7 | 295 |
| (culture) | | | | |
| Blue spruce needles | RNA | 0.11 | 1.5 | 16 |
| (dormant tree) | reagent | | | |
| Blue spruce needles | RNA | 2.7 | 1.4 | 264 |
| (dormant tree) | reagent | | | |
| Blue spruce needles | Trizol | 2.7 | 0.6 | 12 |
| (dormant tree) | | | | |
| Grass | RNA | 1.0 | 1.8 | 463 |
| | reagent | | | |
| Grass | Trizol | 1.0 | 1.8 | 160 |
| Fungal hyphae, | RNA | 10⁹ cells | 1.8 | 274 |
| sense | reagent | | | |
| Fungal hyphae, | RNA | 10⁹ cells | 1.9 | 57 |
| anti-sense | reagent | | | |
| HeLa cells | RNA | 10⁸ cells | 1.8 | 2296 |
| | reagent | | | |

Having now fully described this invention it will be understood to those of ordinary skill in the art that the same can be performed within a wide and equivalent range of conditions, formulations and other parameters without affecting the scope of the invention or any embodiment thereof. All patents, patent applications and publications cited herein are entirely incorporated by reference herein.

What is claimed is:

1. An aqueous RNA isolation reagent, comprising two or more components selected from the group consisting of
(a) at least one nonionic detergent at a concentration of 0.1-1.0% (vol/vol);
(b) at least one chelator at a concentration of 0.02-0.25 M; and
(c) at least one phenol at a concentration of 10%-60% (wgt/vol); and
(d) at least one phenol solubilizer at a concentration of 15%-55% (vol/vol).

2. An RNA isolation reagent according to claim 1, wherein said at least one nonionic detergent is present at a concentration of 0.5-0.8% (vol/vol).

3. An RNA isolation reagent according to claim 3, wherein said nonionic detergent comprises at least one detergent selected from the group consisting of an octylphenoxypoly(oxyethylene)ethanol, an N,N-bis(3-D-gluconamidopropyl)cholamide (BIGCHAP), an decanoyl-N-methylglucamide, an n-decyl α-D-glucopyranoside, an n-decyl β-D-glucopyranoside, an n-decyl β-D-maltopyranoside, a deoxy-BIGCHAP, a digitonin, an n-dodecyl β-D-glucopyranoside, an n-dodecyl α-D-maltoside, an n-dodecyl β-D-maltoside, a heptanoyl-N-methylglucarnide, an n-heptyl β-D-glucopyranoside, an N-heptyl β-D-thioglucopyranoside, an n-hexyl β-D-glucopyranoside, a 1-monooleoyl-rac-glycerol, a nonanoyl-N-methylglucamide, an n-nonyl α-D-glucopyranoside, an n-nonyl β-D-glucopyranoside, an octanoyl-N-methylglucamide, an n-octyl α-D-glucopyranoside, an n-octyl β-D-glucopyranoside, an octyl β-D-thiogalactopyranoside, an octyl β-D-thioglucopyranoside, a polyoxyethylene ester, a polyoxyethylene ether, a polyoxyethylenesorbitan ester, Tween 20, a sorbitan ester, an n-tetradecyl β-D-maltoside, a triton, a tyloxaapol and an n-undecyl β-D-glucopyranoside.

4. An RNA extraction reagent according to claim 3, wherein said non-ionic detergent is a octylphenoxypoly(oxyethylene)ethanol.

5. An RNA extraction reagent according to claim 4, wherein said oetylphenoxy(poly(oxyethylene))ethanol is present at a concentration of about 0.5% (vol/vol).

6. An RNA extraction reagent according to claim 1, wherein said chelator is selected from the group consisting of sodium citrate, EDTA, EGTA, sodium citrate, a citric acid, a salicylic acid or a salt thereof, a tergitol, a phthalic acid, a 2,4 pentanedione, a histidine, a histidinol dihydrochloride, an 8-hydroxyquinoline, an 8-hydroxyquinoline citrate and an o-hydroxyquinone.

7. An RNA extraction reagent according to claim 1, wherein said phenol is a compound according to formula I: 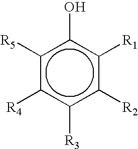
where R₁, R₂, R₃, R4, R₅ are each independently selected from H, alkyl, o-alkyl, halo, acyl and hydroxyl.

8. An RNA extraction reagent according to claim 1, further comprising at least one phenol solubilizer at a concentration of 15%-55% (wgt/vol).

9. An RNA extraction reagent according to claim 8, wherein said phenol solubilizer is selected from the group consisting of a monoalcohol, a diol and a polyol.

10. An RNA extraction reagent according to claim 1, further comprising at least one phenol stabilizer at a concentration of 0.05%-0.2% (wgt/vol).

11. An RNA extraction reagent according to claim 10, wherein said phenol stabilizer is at least one selected from the group consisting of hydroxyquinoline, 8-hydroxyquinoline, 8-hydroxyquinoline citrate, 2,5,7,8-tetramethyl-2-(4′,8′,12′-trimethyltridecyl)-6-chromanol, p-hydroxyquinone, o-hydroxyquinone, citric acid or salt thereof, salicylic acid, ascorbic acid, p-phenylenediamine and n-propylgallate.

12. A kit for isolation of RNA, comprising at least one container, wherein a first container contains at least one RNA extraction reagent according to claim 1.

13. A method for providing cytoplasmic RNA from a sample comprising eukaryotic cells, said method comprising
(a) mixing said sample containing said cells with an RNA extraction reagent according to claim 1 to form a mixture;
(b) adding a haloalkane to the mixture and mixing the resulting organic and aqueous phases;
(c) separating the organic and aqueous phases; and
(d) precipitating cytoplasmic RNA from the aqueous phase obtained in step (c).

14. A method according to claim 13, further comprising
(e) recovering the cytoplasmic RNA from the precipitate obtained in step (d).

15. A method according to claim 14, further comprising
(f) isolating mRNA from said cytoplasmic RNA.

16. A method according to claim 14, wherein said sample is derived from a plant or a plant material.

17. A method according to claim 13, wherein said cells are plant cells.

18. A method according to claim 13, wherein said cells are animal cells.

19. A method according to claim 17, wherein said animal cells are mammalian cells.

20. A method according to claim 13, wherein said cells are insect cells.
